(19) United States
(12) Patent Application Publication
(10) Pub. No.: US 2006/0229710 A1
(43) Pub. Date: Oct. 12, 2006

(54) Vascular graft

(76) Inventors: Thomas O'Brien, County Cork (IE); Michael Thomas Walsh, County Limerick (IE); Timothy M. McGloughlin, County Limerick (IE); Pierce Grace, County Limerick (IE); Siobhan Mary O'Callaghan, County Clare (IE); Paul David Devereux, Dublin (IE)

Correspondence Address: JACOBSON HOLMAN PLLC, 400 SEVENTH STREET N.W., SUITE 600, WASHINGTON, DC, 20004, US

(21) Appl. No.: 11/386,869

(22) Filed: Mar. 23, 2006

Related U.S. Application Data
(60) Provisional application No. 60/664,209, filed on Mar. 23, 2005.

(51) Int. Cl. A61F 2/06

(52) U.S. Cl. 623/1.35

(57) ABSTRACT

A vascular graft (30) comprises a proximal inlet section (31), a first distal section (32) and a second distal section (33). The first distal section (32) and the second distal section (33) are attached to the proximal inlet section (31) at a Y-shaped bifurcation region. In use the proximal inlet section (31) is attached to a first part (34) of a host artery in an end-to-side anastomosis. A second part (35) of the host artery is cut to form a first section (36) of the host artery on a first side of the cut and a second section (37) of the host artery on a second side of the cut. The first distal section (32) is attached to the first section (36) in an end-to-end anastomosis, and the second distal section (33) is attached to the second section (37) in an end-to-end anastomosis. When implanted the graft (30) directs blood flow from the first part (34) of the host artery through the proximal inlet section (31), into the second distal section (33) and into the second section (37) of the host artery along a single flow path.

### INTRODUCTION

This invention relates to a vascular graft.

Referring to Fig. A, a conventional end-to-side bypass graft is shown, and Fig. B shows simulated flow patterns. The graft is sutured to the host artery at its distal end. There is a recirculation region, and the blood impinges on the artery bed. This may result in abnormal wall shears which may contribute to disease formation.

This invention is aimed at providing a vascular graft which addresses at least some of these problems.

### STATEMENTS OF INVENTION

According to the invention there is provided a vascular graft comprising:
a proximal section;
a first distal section having an end configured for end-to-end anastomosis with a first section of a host artery, and
a second distal section having an end configured for end-to-end anastomosis with a second section of the host artery.

In one embodiment of the invention the longitudinal axis of the first distal section is out of alignment with the longitudinal axis of the second distal section. The angle subtended between the longitudinal axis of the first distal section and the longitudinal axis of the second distal section may be an obtuse angle. The angle subtended between the longitudinal axis of the first distal section and the longitudinal axis of the second distal section may be an acute angle. In one case the longitudinal axis of the second distal section is substantially aligned with the longitudinal axis of the proximal section.

In one embodiment the first distal section and the second distal section are attached to the proximal section at a bifurcation region. Preferably the proximal section, the first distal section, and the second distal section are interconnected at a substantially Y-shaped junction.

In one embodiment at least two of the sections are formed integrally. The second distal section may be an outlet for flow from the proximal section. The second distal section may be formed integrally with the proximal section. The first distal section may be configured for substantially small or nil flowrate. In one case the proximal section comprises a proximal inlet section.

In another embodiment at least one of the distal sections is substantially curved and/or is configured to be substantially curved. Preferably the curved distal section is curved and/or is configured to be curved through greater than 90°.

In one case the first distal section is configured for anastomosis with a first section of a host artery on a first side of a cut through the host artery, and the second distal section is configured for anastomosis with a second section of the host artery on a second side of the cut through the host artery.

The end of the distal section may be configured for butt anastomosis with a section of a host artery. The end of the distal section may be configured for lap anastomosis with a section of a host artery.

In one embodiment the graft is configured to direct flow between a first part of a host artery and a second part of a host artery along a single flow path.

In another aspect of the invention there is provided a method of performing a surgical procedure, the method comprising the steps of:—
providing a vascular graft comprising a first distal section and a second distal section;
connecting an end of the first distal section to a first section of a host artery with an end-to-end anastomosis; and
connecting an end of the second distal section to a second section of the host artery with an end-to-end anastomosis.

In one embodiment of the invention the method comprises the step of moving the first section of the host artery relative to the second section of the host artery to move the longitudinal axis of the first section out of alignment with the longitudinal axis of the second section. The first section of the host artery may be substantially parallel to the second section of the host artery.

The method may comprise the step of cutting the host artery to form the first section of the host artery on a first side of the cut and the second section of the host artery on a second side of the cut. The method may comprise the step of cutting the host artery to form the first section of the host artery on a first side of the cut and the second section of the host artery on a second side of the cut without removing substantially any portion of the host artery.

In one case the vascular graft directs flow between a first part of the host artery and a second part of the host artery along a single flow path.

The method may comprise the step of curving at least part of a distal section before connecting the end of the distal section to the section of the host artery to minimise displacement of the host artery. The distal section may be curved through greater than 90°.

According to a further aspect, the invention provides a method of performing a surgical procedure using a vascular graft of the invention, the method comprising the steps of:
cutting a host artery and separating the ends exposed by the cut;
suturing the end of each distal section to an exposed artery end; and
attaching the proximal section to a vessel for supply of blood through the proximal section to the second distal section.

According to the invention, there is provided a vascular graft comprising:
a proximal inlet section;
a first distal section having an end for an end-to-end anastomosis with a host artery, and
a second distal section having an end for an end-to-end anastomosis with the host artery.

In one embodiment, the proximal inlet section, the first distal section, and the second distal section are interconnected at a Y junction.

In another embodiment, the sections are integral.

In a further embodiment, the second distal section is an outlet for flow from the proximal inlet section.

In one embodiment, the second distal section is integral with the proximal inlet section.

In another embodiment, the first distal section is for small or nil flowrate.

In another aspect, the invention provides a method of performing a surgical procedure using a graft of any preceding claim, comprising the steps of:
cutting a host artery and separating the ends exposed by the cut;
suturing the end of each distal section to an exposed artery end; and
attaching the proximal inlet section to a vessel for supply of blood through the proximal inlet section to the second distal section.

### BRIEF DESCRIPTION OF THE DRAWINGS

The invention will be more clearly understood from the following description of some embodiments thereof, given by way of example only, with reference to the accompanying drawings, in which:—

FIGS. 1 and 2 are diagrams of a graft of the invention;

FIG. 3 is a diagram of simulated flow pattern in the graft;

FIG. 4 is a diagram of another graft of the invention;

FIG. 5 are velocity vector diagrams for the graft of FIG. 4;

FIG. 6 is a series of diagrams showing surgical implantation of the graft of FIG. 4;

FIG. 7 is a diagram of an alternative graft of the invention;

FIG. 8 is a schematic illustration of another vascular graft according to the invention;

FIG. 9 is an enlarged view of part of the vascular graft of FIG. 8;

FIG. 10 is an enlarged view of part of the vascular graft of FIG. 8; and

FIG. 11 is an enlarged view of part of another vascular graft according to the invention.

### DETAILED DESCRIPTION

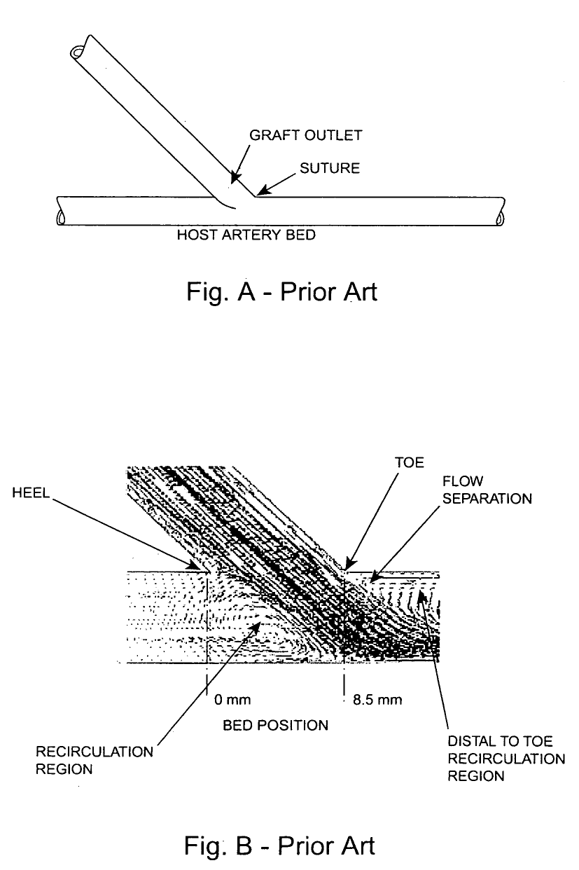

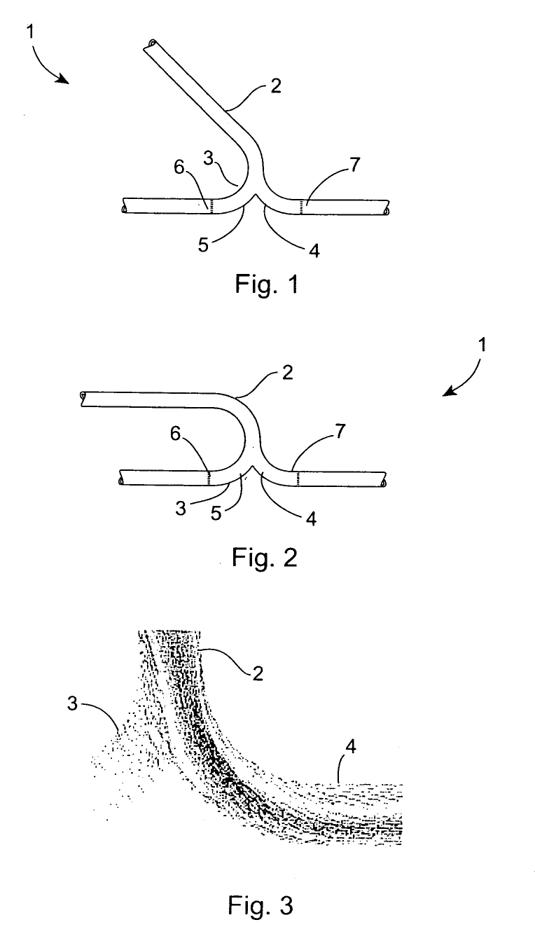

Referring to FIG. 1 a vascular graft 1 comprises a proximal section 2 and integral distal sections 3 and 4. The sections 2, 3, and 4 are integrally interconnected at a Y junction 5. The graft 1 is sutured to the host artery at end-to-end anastomoses 6 and 7. The configuration of the graft 1 in practice for many surgical procedures is shown in FIG. 2.

Referring to FIG. 3, flow is through the proximal section 2 and out through the distal section 4. There is little or no flow in the distal section 3 as the host artery is blocked beyond the anastomosis 6.

The bifurcation 5 does not result in any sudden changes in blood flow direction, and as a result, abnormal wall shears and stresses do not occur on the host artery, the flow recirculation region is reduced in size and magnitude, and the toe flow separation region is eliminated.

An additional benefit is that the graft 1 removes a section of artery completely. As the graft bifurcation 5 is synthetic, disease may not form at the artery bed as in the prior art case, as it has been removed. In addition, the excision of the section of artery is useful in stent revision surgery, where the stent becomes occluded and must be removed, together with the host artery surrounding it. The diseased artery section can be excised and the natural bifurcation graft 1 implanted.

An additional advantage of the invention is that the graft 1 may be manufactured using commercially available vascular graft materials. The natural bifurcation can be cut and assembled together using tubular vascular grafts. This allows the surgeon to tailor each graft to the patient requirements. It is also possible to manufacture a range of grafts with natural bifurcated ends.

The CFD investigations were validated experimentally using in vitro models of end-to-side anastomoses. The flow patterns through the junction were recorded using laser Doppler anemometry (LDA) and compared to the CFD results to establish the accuracy of the numerical modelling procedure. The results were found to compare within acceptable limits, meaning the CFD models were suitable for in vitro testing and experimentation.

Within the in vitro environment, it has been established that the natural bifurcation graft produces less abnormal junction haemodynamics than when compared to conventional bypass grafts.

Variations on the embodiment described will be appreciated by those skilled in the art. The graft may be a prosthetic, flexible graft, or an autologous, compliant vein graft or an autologous, compliant artery graft. The graft may have a tissue-engineered bifurcation. The bifurcation may be assembled through suturing or bonding of commercially available tubular sections. It may alternatively be prefabricated as a whole. The angles may vary, and the lengths of the graft inlet section 2 and the two outlet sections 3, 4 may vary. The configurations of the tubular sections may vary.

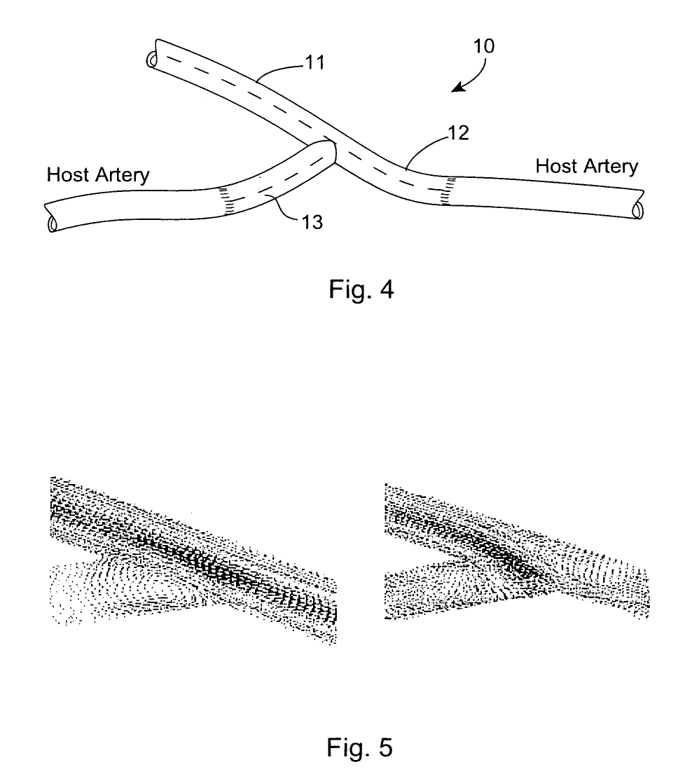

Referring to FIG. 4 an alternative graft 10 is shown. Again, there is a proximal section 11, a distal section 12, and another distal section 13. The angle between the distal sections 12, 13 is greater in this embodiment. In particular an obtuse angle is subtended between the longitudinal axis of the first distal section 13 and the longitudinal axis of the second distal section 12.

Again, the graft 10 eliminates the disease prone artery bed. It replaces the prior art end-to-side anastomosis with end-to-end anastomosis, which has higher patency rates. Flow disturbance in the graft 10 is minimised as is flow disturbance into the host artery, due to the streamlined nature of the sections 11 and 12.

Correctly designed and implanted, it would be possible to avoid flow disturbance which may be sensed by the host arteries. Computational fluid dynamic results of the junction haemodynamics for a typical femoral bypass procedure suggest that within 20 mm after leaving the junction, the flow returns to being fully developed (FIG. 5).

FIG. 5 shows the effects of no flow and flow (50:50) in the section 13. The flow is seen to fully develop within 20 mm from leaving the junction. This suggests that 20 mm long graft sections after the junction are sufficient to return the blood flow patterns to their normal state.

The graft 10 may be manufactured by the surgeon using commercially available tubular bypass grafts. It may also be prefabricated to optimised designs. The angle between the proximal leg 13 and the main graft conduit 11 may be varied. For low proximal flow-rates, the calibre of the proximal leg 13 may be reduced.

The proximal junction section may also be made using autologous vein with a prosthetic graft leading up to an autologous vein junction, proximal leg and distal vein conduit.

### EXAMPLE

The Y-graft may be used to create an above- or below-knee femoro-popliteal bypass. The common femoral artery is exposed in the groin via a longitudinal groin incision; the common, superficial and profunda femoris arteries are exposed and slings are placed around each artery. The popliteal artery is exposed via a longitudinal incision on the side of the thigh above the knee for an above knee bypass or the medial side of the calf below the knee for a below knee bypass. The popliteal artery is dissected and slings placed around it proximally and distally. Heparin 5,000 i.u. is administered intravenously and the arteries are clamped.

The distal anastomoses to the popliteal artery are created first. The popliteal artery is transected and the cut ends are turned up into the wound. One limb of the Y-graft is anastomosed in a standard end-to-end fashion to the ‘proximal’ cut end of the popliteal artery using 5/0 or 6/0 prolene sutures. The other limb of the Y-graft is anastomosed to the ‘distal’ cut end of the popliteal artery using 5/0 or 6/0 prolene sutures. The proximal end of the Y-graft is then tunnelled to the groin wound and is anastomosed to the common femoral artery in a standard end-to-side fashion using 5/0 or 6/0 prolene sutures. The clamps are removed and the wounds are closed over suction drains.

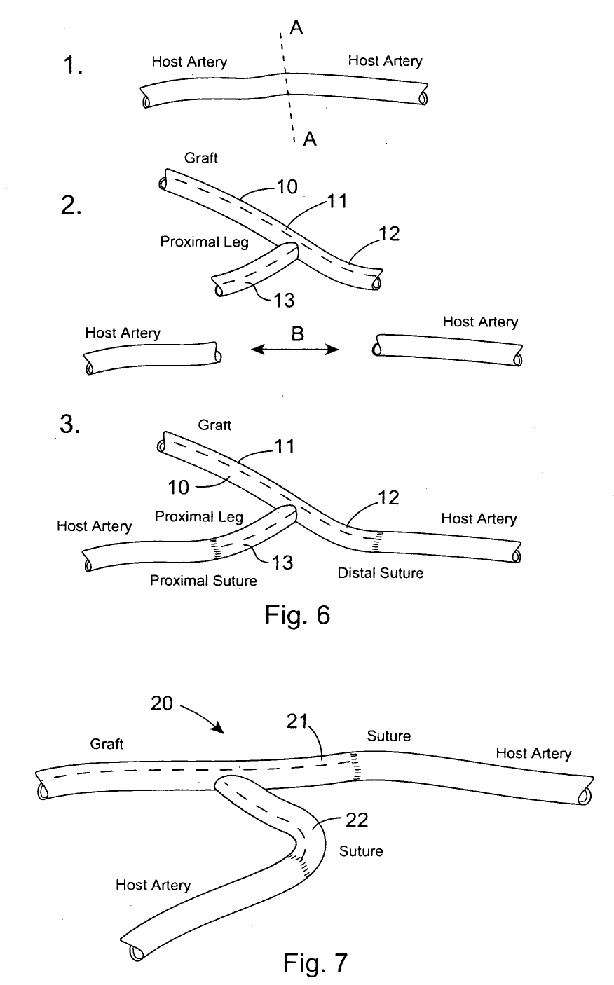

Referring to FIG. 6 the implantation of the Y-graft 10 differs from the conventional anastomoses, in that two end-to-end sutures are used to attach it to the host artery, rather than the single, end-to-side anastomosis used in the conventional anastomoses. In one procedure:
1. The host artery is cut through its cross-section (A-A).
2. The two artery ends are separated, exposing a gap between them (B).
3. The Y-graft 10 is brought into position. An end-to-end suture is used to attach one leg to the host artery upstream of the anastomosis and an end-to-end suture is used to attach the other leg to the host artery downstream of the anastomosis.

If the gap between the artery ends is insufficient, a length of artery may be excised. Various configurations of the junction are possible under the same implantation principle.

The Y-graft may also have applications in performing femoro-tibial bypasses, coronary artery bypass and carotid artery reconstruction.

The invention is not limited to the embodiments hereinbefore described, with reference to the accompanying drawings, but may be varied in construction and detail. For example, the angles and/or diameters and/or section lengths may vary according to the precise intended end use. For example, FIG. 7 shows a further embodiment, in which the angle between the two distal sections 21 and 22 in a graft 20 is less than for the other embodiments.

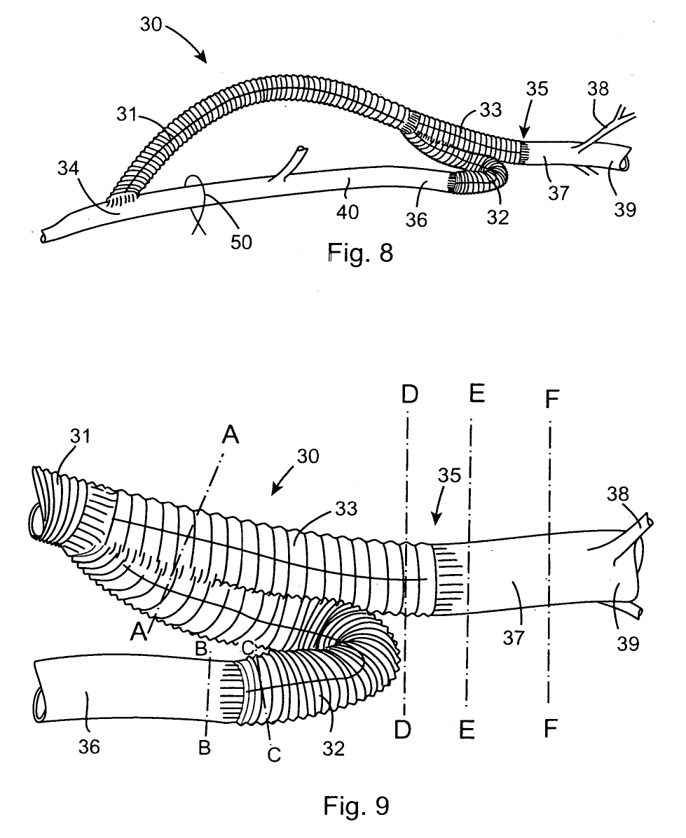

Referring to FIGS. 8 to 10 there is illustrated another vascular graft 30 according to the invention, which is similar to the vascular grafts described previously with reference to FIGS. 1 to 7.

In this case the graft 30 comprises a proximal inlet section 31, a first distal section 32 and a second distal section 33.

In use the proximal inlet section 31 is attached to a first part 34 of a host artery in an end-to-side anastomosis. A second part 35 of the host artery downstream of the first part 34 is cut to form a first section 36 of the host artery on a first side of the cut and a second section 37 of the host artery on a second side of the cut. The first section 36 is moved relative to the second section 37 to move the longitudinal axis of the first section 36 out of alignment with the longitudinal axis of the second section 37. The first distal section 32 is attached to the first section 36 in an end-to-end anastomosis, and the second distal section 33 is attached to the second section 37 in an end-to-end anastomosis.

As illustrated in FIG. 8, the first distal section 32 and the second distal section 33 are attached to the proximal inlet section 31 at a Y-shaped bifurcation region. In this case the proximal inlet section 31, the first distal section 32 and the second distal section 33 are formed integrally. The longitudinal axis of the first distal section 32 is out of alignment with the longitudinal axis of the second distal section 33 with an acute angle subtended between the longitudinal axes. In addition the longitudinal axis of the second distal section 33 is substantially aligned with the longitudinal axis of the proximal inlet section 31 for smooth blood flow from the proximal inlet section 31 into the second distal section 33. The flow rate through the first distal section 32 is substantially small or nil.

As illustrated in FIG. 9, the first distal section 32 curves through greater than 90° from the end of the first section 36 of the host artery to the proximal inlet section 31. The first distal section 32 may have this curved shape pre-formed or may be of a flexible/malleable material to enable the curve to be formed during implantation.

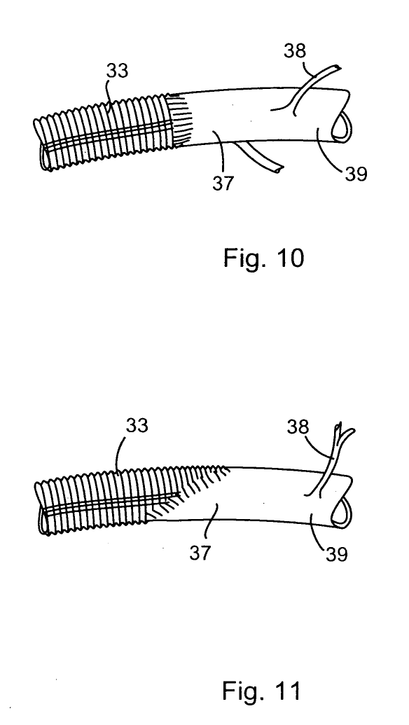

The first distal section 32 may be attached to the first section 36 of the host artery with a butt anastomosis as illustrated in FIG. 10, or alternatively with a lap anastomosis as illustrated in FIG. 11. Similarly the second distal section 33 may be attached to the second section 37 of the host artery with a butt anastomosis, or alternatively with a lap anastomosis.

When implanted the graft 30 directs blood flow from the first part 34 of the host artery through the proximal inlet section 31, into the second distal section 33 and into the second section 37 of the host artery along a single flow path.

In further detail, FIG. 8 illustrates the main graft conduit 31, the distal graft leg 33, the distal junction end-to-end anastomosis, the collateral artery 38, the tie-off ligature representing the blockage 50, the proximal host artery 40, the proximal junction end-to-end anastomosis, the proximal graft leg 32, the distal host artery 39.

In use, the host artery is cut to accommodate the device 30. There is no requirement to remove a portion of the host artery, due to the advantage of the looping around of the proximal graft leg 32. The host artery exposed ends may be shifted out of line with each other. Alternatively, a small portion of the artery may be resected to create space for the two end-to-end anastomoses.

The graft legs 32, 33 may be cut as required according to a number of constraints. The proximal graft leg 32 should be looped long enough to prevent kinking. The length of the legs 32 is at the discretion of the surgeon. The distal graft leg 33 should in this particular case be at least 20 mm long to enable flow recovery. A ligature 50 may be used to tie off the proximal host artery 40 to simulate blockage.

FIG. 9 illustrates a number of cross-sections.

Junction

A-A: Random section through the junction.

Proximal Host Artery

B-B: 1 to 2 mm upstream from proximal junction end-to-end anastomosis

Proximal Graft Leg

C-C: 1 to 2 mm downstream from proximal junction end-to-end anastomosis

Distal Graft Leg

D-D: 5 mm upstream from distal junction end-to-end anastomosis

Distal Host Artery

E-E: 1 to 2 mm downstream from distal junction end-to-end anastomosis

F-F: 10 mm downstream from distal junction end-to-end anastomosis

1. A vascular graft comprising:
a proximal section;
a first distal section having an end configured for end-to-end anastomosis with a first section of a host artery, and
a second distal section having an end configured for end-to-end anastomosis with a second section of the host artery.

2. A graft as claimed in claim 1 wherein the longitudinal axis of the first distal section is out of alignment with the longitudinal axis of the second distal section.

3. A graft as claimed in claim 1 wherein the angle subtended between the longitudinal axis of the first distal section and the longitudinal axis of the second distal section is an obtuse angle.

4. A graft as claimed in claim 1 wherein the angle subtended between the longitudinal axis of the first distal section and the longitudinal axis of the second distal section is an acute angle.

5. A graft as claimed in claim 1 wherein the longitudinal axis of the second distal section is substantially aligned with the longitudinal axis of the proximal section.

6. A graft as claimed in claim 1 wherein the first distal section and the second distal section are attached to the proximal section at a bifurcation region.

7. A graft as claimed in claim 6, wherein the proximal section, the first distal section, and the second distal section are interconnected at a substantially Y-shaped junction.

8. A graft as claimed in claim 1 wherein at least two of the sections are formed integrally.

9. A graft as claimed in claim 1 wherein the second distal section is an outlet for flow from the proximal section.

10. A graft as claimed in claim 1 wherein the second distal section is formed integrally with the proximal section.

11. A graft as claimed in claim 1 wherein the first distal section is configured for substantially small or nil flowrate.

12. A graft as claimed in claim 1 wherein the proximal section comprises a proximal inlet section.

13. A graft as claimed in claim 1 wherein at least one of the distal sections is substantially curved and/or is configured to be substantially curved.

14. A graft as claimed in claim 13 wherein the curved distal section is curved and/or is configured to be curved through greater than 90°.

15. A graft as claimed in claim 1 wherein the first distal section is configured for anastomosis with a first section of a host artery on a first side of a cut through the host artery, and the second distal section is configured for anastomosis with a second section of the host artery on a second side of the cut through the host artery.

16. A graft as claimed in claim 1 wherein the end of the distal section is configured for butt anastomosis with a section of a host artery.

17. A graft as claimed in claim 1 wherein the end of the distal section is configured for lap anastomosis with a section of a host artery.

18. A graft as claimed in claim 1 wherein the graft is configured to direct flow between a first part of a host artery and a second part of a host artery along a single flow path.

19. A method of performing a surgical procedure, the method comprising the steps of:—
providing a vascular graft comprising a first distal section and a second distal section;
connecting an end of the first distal section to a first section of a host artery with an end-to-end anastomosis; and
connecting an end of the second distal section to a second section of the host artery with an end-to-end anastomosis.

20. A method as claimed in claim 19 wherein the method comprises the step of moving the first section of the host artery relative to the second section of the host artery to move the longitudinal axis of the first section out of alignment with the longitudinal axis of the second section.

21. A method as claimed in claim 19 wherein the first section of the host artery is substantially parallel to the second section of the host artery.

22. A method as claimed in claim 19 wherein the method comprises the step of cutting the host artery to form the first section of the host artery on a first side of the cut and the second section of the host artery on a second side of the cut.

23. A method as claimed in claim 19 wherein the method comprises the step of cutting the host artery to form the first section of the host artery on a first side of the cut and the second section of the host artery on a second side of the cut without removing substantially any portion of the host artery.

24. A method as claimed in claim 19 wherein the vascular graft directs flow between a first part of the host artery and a second part of the host artery along a single flow path.

25. A method as claimed in claim 19 wherein the method comprises the step of curving at least part of a distal section before connecting the end of the distal section to the section of the host artery to minimise displacement of the host artery.

26. A method as claimed in claim 25 wherein the distal section is curved through greater than 90°.

27. A method of performing a surgical procedure using a vascular graft of claim 1 the method comprising the steps of:—
cutting a host artery and separating the ends exposed by the cut;
suturing the end of each distal section to an exposed artery end; and
attaching the proximal section to a vessel for supply of blood through the proximal section to the second distal section.
